(19) United States
(12) Patent Application Publication
(10) Pub. No.: US 2023/0364280 A1
(43) Pub. Date: Nov. 16, 2023

(54) SYSTEM AND METHOD FOR SUPPRESSING BACTERIAL OR VIRAL GROWTH USING A COMBINATION OF LIGHTS

(71) Applicant: Ig Soo Kwon, San Ramon, CA (US)

(72) Inventor: Ig Soo Kwon, San Ramon, CA (US)

(21) Appl. No.: 18/308,045

(22) Filed: Apr. 27, 2023

Related U.S. Application Data
(60) Provisional application No. 63/342,517, filed on May 16, 2022.

(51) Int. Cl. A61L 2/08; A61L 2/24

(52) U.S. Cl. CPC A61L 2/084; A61L 2/085; A61L 2/24

(57) ABSTRACT

A system includes a power supply and a light coupled to the power supply and including a plurality of light-emitting diodes (LEDs). The plurality of LEDs includes a first LED that has a first wavelength and a second LED that has a second wavelength that is different from the first wavelength. A first number of the first LED is greater than a second number of the second LED.

### CROSS-REFERENCE TO RELATED APPLICATION(S)

This application claims the benefits of and priority to U.S. Provisional Patent Application Ser. No. 63/342,517 filed May 16, 2022, the disclosure of which is incorporated herein by reference in its entirety.

### TECHNICAL FIELD

The present disclosure relates generally to a system and method for suppressing bacterial or viral growth, in particular, a system and method for suppressing bacterial or viral growth using a combination of lights having at least two different wavelength spectra.

### BACKGROUND

Due to the availability of antibiotics, the morbidity and mortality derived from bacterial infectious diseases have been declined substantially. Although the availability of diverse antibiotics saved millions or even billions of lives over the past several decades, there are still many bacterial or viral infection-related deaths or out-breaks through media every year. For example, respiratory diseases-related complications such as sinusitis, otitis, and tonsillopharyngitis have been frequent issues, especially to children. Because of the continuous rise of antibiotics-resistant bacteria, millions of people in the United States alone acquire bacteria-based infectious diseases, and more than 35,000 people lose their lives every year as a direct consequence of resistant bacterial or viral infections. Therefore, there is a high demand to apply a method to sanitize or disinfect harmful microbes in the surroundings of our daily lives. Due to the recent COVID-19 pandemic, numerous studies have launched to find inexpensive yet effective solutions to reduce or prevent potential infections using light-emitting diode (LED)-based lights.

### SUMMARY

According to one embodiment, a system includes a power supply and a light coupled to the power supply and including a plurality of light-emitting diodes (LEDs). The plurality of LEDs includes a first LED that has a first wavelength and a second LED that has a second wavelength that is different from the first wavelength. A first number of the first LED is greater than a second number of the second LED.

According to another embodiment, a method for sanitizing a surface of a target object includes emitting a combined light of a first wavelength and a second wavelength. A first light intensity of a first LED that emits the first wavelength is greater than a second light intensity of a second LED that emits the second wavelength.

The above and other preferred features, including various novel details of implementation and combination of events, will now be more particularly described with reference to the accompanying figures and pointed out in the claims. It will be understood that the particular systems and methods described herein are shown by way of illustration only and not as limitations.

As will be understood by those skilled in the art, the principles and features described herein may be employed in various and numerous embodiments without departing from the scope of the present disclosure.

### BRIEF DESCRIPTION OF THE DRAWINGS

The accompanying drawings, which are included as part of the present specification, illustrate the presently preferred embodiment and together with the general description given above and the detailed description of the preferred embodiment given below serve to explain and teach the principles described herein.

FIG. 1 shows an example of the present system, according to one embodiment;

FIG. 2 shows an example of the present system, according to another embodiment;

FIG. 3 shows an example of the present system, according to yet another embodiment;

FIG. 4 shows an example of the present system, according to yet another embodiment;

FIG. 5 shows an example of a light, according to one embodiment;

FIG. 6 shows an example of a light, according to another embodiment;

FIG. 7 shows test results of LED light exposure of 4V, 3V-1IR, 2V-21R, 1V-31R, and 4R to E. coli at a distance of 0.5 meter in 6 hours;

FIG. 8 shows test results of LED light exposure of 4V, 3V-1IR, 2V-21R, 1V-31R, and 4R to E. coli at a distance of 1 meter in 12 hours;

FIG. 9 shows test results of LED light exposure of 4V, 3V-1IR, 2V-21R, 1V-31R, and 4R to E. coli at a distance of 2 meters in 12 hours.

FIG. 10 shows test results of LED light exposure of 4V, 3V-1IR, 2V-21R, 1V-31R, and 4R to Staphylococcus aureus at a distance of 0.5 meter in 6 hours;

FIG. 11 shows test results of LED light exposure of 4V, 3V-1IR, 2V-21R, 1V-31R, and 4R to Staphylococcus aureus at a distance of 1 meter in 12 hours;

FIG. 12 shows test results of LED light exposure of 4V, 3V-1IR, 2V-21R, 1V-31R, and 4R to Staphylococcus aureus at a distance of 2 meters in 24 hours;

FIG. 13 shows test results of LED light exposure of 4V, 3V-1IR, 2V-21R, 1V-31R, and 4R to MDR-E. coli at a distance of 0.5 meter in 6 hours;

FIG. 14 shows test results of LED light exposure of 4V, 3V-1IR, 2V-2IR, 1V-3IR, and 4R to MDR-E. coli at a distance of 1 meter in 12 hours;

FIG. 15 shows test results of LED light exposure of 4V, 3V-1IR, 2V-2IR, 1V-3IR, and 4R to MDR-E. coli at a distance of 2 meters in 24 hours.

FIG. 16 shows test results of LED light exposure of 4V, 3V-1IR, 2V-2IR, 1V-3IR, and 4R to MDR-Salmonella enterica at a distance of 0.5 meter in 6 hours;

FIG. 17 shows test results of LED light exposure of 4V, 3V-1IR, 2V-2IR, 1V-3IR, and 4R to MDR-Salmonella enterica at a distance of 1 meter in 12 hours;

FIG. 18 shows test results of LED light exposure of 4V, 3V-1IR, 2V-21R, 1V-31R, and 4R to MDR-Salmonella enterica at a distance of 2 meters in 24 hours;

FIG. 19 shows test results of LED light exposure of 4V, 3V-1IR, 2V-21R, 1V-31R, and 4R to MDR-Staphylococcus aureus at a distance of 0.5 meter in 6 hours;

FIG. 20 shows test results of LED light exposure of 4V, 3V-1IR, 2V-21R, 1V-31R, and 4R to MDR-Staphylococcus aureus at a distance of 1 meter in 12 hours;

FIG. 21 shows test results of LED light exposure of 4V, 3V-1IR, 2V-21R, 1V-31R, and 4R to MDR-Staphylococcus aureus at a distance of 2 meters in 24 hours;

FIG. 22 shows test results of LED light exposure of 4V, 3V-1IR, 2V-21R, 1V-31R, and 4R to COVID-19 viral particles at a distance of 0.5 meter in 6 hours; and

FIG. 23 shows a relative difference in RNA copies in reaction for the same test results of FIG. 22.

The figures are not necessarily drawn to scale and elements of similar structures or functions are generally represented by like reference numerals for illustrative purposes throughout the figures. The figures are only intended to facilitate the description of the various embodiments described herein. The figures do not describe every aspect of the teachings disclosed herein and do not limit the scope of the claims.

### DETAILED DESCRIPTION

Each of the features and teachings disclosed herein can be utilized separately or in conjunction with other features and teachings to provide a method and system for suppressing bacterial or viral growth using a combination of lights having at least two different wavelength spectra. Representative examples utilizing many of these additional features and teachings, both separately and in combination, are described in further detail with reference to the attached figures.

This detailed description is merely intended to teach a person of skill in the art further details for practicing aspects of the present teachings and is not intended to limit the scope of the claims.

Therefore, combinations of features disclosed above in the detailed description may not be necessary to practice the teachings in the broadest sense, and are instead taught merely to describe particularly representative examples of the present teachings.

In the description below, for purposes of explanation only, specific nomenclature is set forth to provide a thorough understanding of the present disclosure. However, it will be apparent to one skilled in the art that these specific details are not required to practice the teachings of the present disclosure.

Some portions of the detailed descriptions herein are presented in terms of algorithms and symbolic representations of operations on data bits within a computer memory. These algorithmic descriptions and representations are used by those skilled in the data processing arts to effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the below discussion, it is appreciated that throughout the description, discussions utilizing terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Moreover, the various features of the representative examples and the dependent claims may be combined in ways that are not specifically and explicitly enumerated in order to provide additional useful embodiments of the present teachings. It is also expressly noted that all value ranges or indications of groups of entities disclose every possible intermediate value or intermediate entity for the purpose of an original disclosure, as well as for the purpose of restricting the claimed subject matter. It is also expressly noted that the dimensions and the shapes of the components shown in the figures are designed to help to understand how the present teachings are practiced, but not intended to limit the dimensions and the shapes shown in the examples.

The present disclosure describes a system and method for suppressing bacterial or viral growth, thereby preventing infectious diseases that may be derived from bacteria, virus, fungus, or other microorganisms. The present system and method combines a first light source in a first wavelength spectrum (e.g., a violet light in a wavelength in 400-450 nm) and a second light source in a second wavelength spectrum (e.g., an infrared light in a wavelength in 700-1000 nm). For example, the first light source has 405 nm wavelength, and the second light source has 850 nm wavelength. In another embodiment, the combined light used herein may include a first light in a visible light spectrum (a violet light) and a second light in an infrared light spectrum. However, it is understood that the wavelengths of the first and second light sources and their spectrum bands are not limited thereto, and various wavelengths in different light spectrum bands with similar effect as understood by the present disclosure may be used without deviating from the scope of the present disclosure.

The present system and method focuses on cleaning, sanitizing, and/or disinfecting bacterial, fungal, or viral infection on a variety of surfaces and in spaces where people routinely spend many hours a day or a week. The United States Environmental Protection Agency provides a guideline on products that disinfect, sanitize, and clean surfaces, and the differences between them. The present system and method relates to a safe non-contact, non-chemical-based treatments and utilizes a combination of lights having at least two wavelength spectra for cleaning, sanitizing, and/or disinfecting bacterial, fungal, or viral infection. The present system and method provides a safe, cost-effective, and versatile way of cleaning, sanitizing, and/or disinfecting surfaces and spaces and maintains sterile conditions in living spaces.

As used herein, a “combined light” may refer to a light having wavelength of at least two or more wavelength spectra, for example, but not limited to, a light including a first light (a violet light) in the wavelength spectrum of 400-450 nm (e.g., 405 nm) and a second light (an infrared light) in the wavelength spectrum of 700-1000 nm (e.g., 850 nm). The ratio of the first and second lights may be user-adjustable based on an operating condition and a user-specific application. For example, the combined light may have a ratio of 3 to 1 that may be dynamically changeable. More specifically, the ratio of the violet light with respect to the infrared light may be greater than one in a case where a power rating of the violet and infrared lights is substantially similar to each other.

The wavelengths and the ratio may be suitably determined and adjusted based on an application and a type of pathogenic bacteria including multiple drug resistant (MDR) strains.

The term of a “violet light” refers to a light in a wavelength spectrum of 400-450 nm (e.g., 405 nm) at a room temperature, in a cold room, or a refrigerator, so that the light effect may be derived from the violet light exclusively without taking into account the thermal effect caused by the infrared light (e.g., 850 nm) that may generate mild heat causing dehydration.

A violet light (herein also referred to as Violet or V) having a wavelength in a range of 400-415 nm may be more effective in suppressing bacterial growth than a blue light having a wavelength in a range of 420-460 nm. In one embodiment, the present system and method uses a violet light having a wavelength of 405 nm in addition to an infrared light (herein also referred to as Infrared or IR).

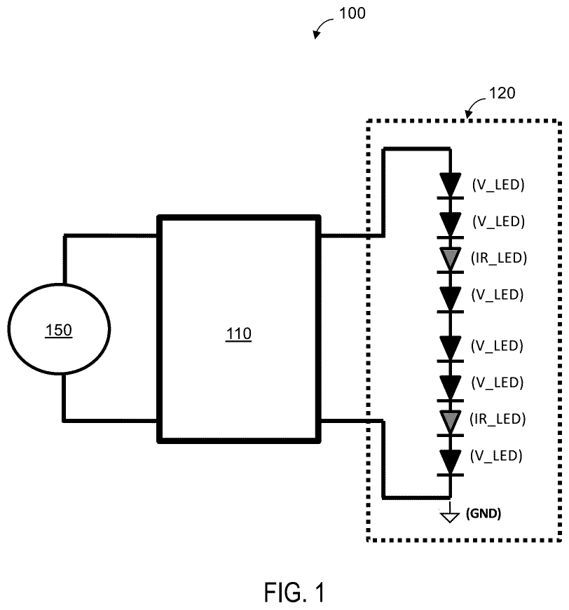

FIG. 1 shows an example of the present system, according to one embodiment. The system 100 includes a power supply 110 and an array of light emitting diode (LED) lighting 120. The array of LED lighting 120 may include both the violet light LED (herein also referred to as V_LED) and the infrared light LED (herein also referred to as IR_LED). In one embodiment, each of a light source may include both the violet light LED and the infrared light LED, and the violet light LED and the infrared light LED may be selectively turned on or off based on a user application. In this case, the system 100 may have a switch allowing a user to selectively turn on or off each of the violet light and the infrared light.

The power supply 110 may supply alternating current (AC) voltage (e.g., 220 VAC) or a direct current (DC) voltage (e.g., 22.4 VDC) depending on a configuration of the system 100 and/or a user application. The power supply 110 may be coupled to an external AC power source 150 or a DC battery (not shown) and supply a DC voltage to the array of LED lighting 120.

As a non-limiting example, the violet light LED has 405 nm wavelength, a power rating of 3 watts, a forward voltage 3.2 V, and the forward current of 400-500 mA, and the infrared light LED has 850 nm wavelength, a power rating of 3 watts, a forward voltage 1.6 V, and the forward current of 400-500 mA. In the example shown in FIG. 1, the array of LED lighting 120 includes six violet lights and two infrared lights at a ratio of 3 to 1 (also referred to as 3V 1IR), and the two infrared lights are arranged to be separate from each other by three violet lights among the six violet lights. However, it is understood that the actual arrangement, the ratio as well as the actual number of the violet light LED and the infrared light LED may be conveniently varied depending on the application. For example, the number of the violet and infrared light LEDs may be increased while maintaining their ratio for a high-power application.

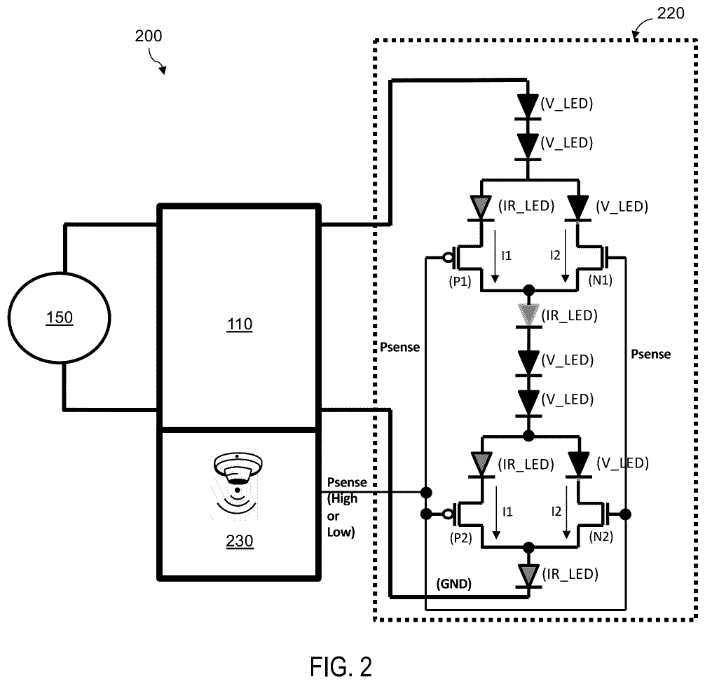

FIG. 2 shows an example of the present system, according to another embodiment. The system 200 includes a power supply 110, an array of light emitting diode (LED) lighting 220, and a distance sensor 230. In the present example, the array of LED lighting 220 includes six violet lights and four infrared lights. The distance sensor 230 may sense a distance from a light source to a target object (not shown) and generate a distance sensing signal Psense. The distance sensing signal Psense may be high if the sensed distance is greater than a certain threshold distance (e.g., 1 meter) or low when the sensed distance is less than the threshold distance.

When the distance sensing signal Psense is low (e.g., the sensed distance is less than 1 meter), switches P1 and P2 are turned on and the switches N1 and N2 are turned off, therefore current I1 flows while current I2 is off. This effectively connects two violet light LEDs, two infrared light LEDs, two more violet light LEDs, and two infrared light LEDs in series configuring the array of LED lighting 220 as having a ratio of 1 to 1 (e.g., 1V 1IR, 2V 2IR, or 4V 4IR). When the distance sensing signal Psense is high (e.g., the sensed distance is greater than 1 meter), switches P1 and P2 are turned off and the switches N1 and N2 are turned on, therefore current I2 flows while current I1 is off. This effectively connects three violet light LEDs, one infrared light LED, three more violet light LEDs, and one infrared light LED in series configuring the array of LED lighting 220 as having a ratio of 3 to 1 (e.g., 3V 1IR). Although the present example shows the configurability between 1 to 1 ratio and 3 to 1 ratio, it is understood that the system 200 is capable of dynamically switching between at least two different ratios of the violet light LEDs and the infrared light LEDs using the distance sensor 230.

According to some test results, the effectiveness of the infrared light may diminish at a longer distance, therefore it may be beneficial to increase the relative ratio of the infrared light with respect to the violet light for a long-distance application to increase the effectiveness of the cleaning, sanitization, and disinfection of the system 200.

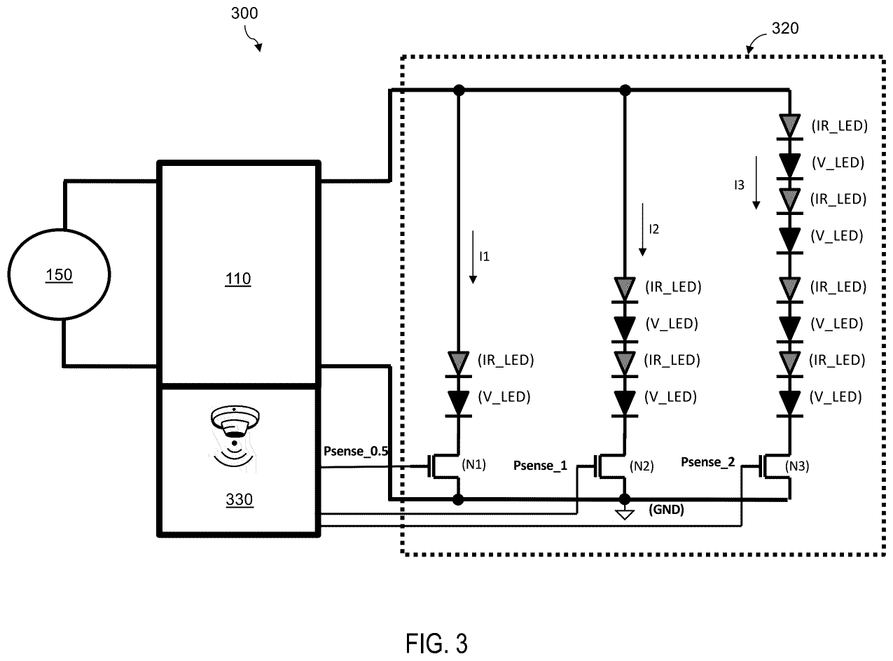

FIG. 3 shows an example of the present system, according to yet another embodiment. The system 300 includes a power supply 110, an array of light emitting diode (LED) lighting 320, and a distance sensor 330. In the present example, the array of LED lighting 320 includes seven violet lights and seven infrared lights. The distance sensor 330 may sense a distance from a light source to a target object and generate a plurality of distance sensing signals Psense_0.5, Psense_1, and Psense_2. For example, the distance sensor 330 may generate the distance sensing signal Psense_0.5 to be high and Psense_1 and Psense_2 to be low if the sensed distance is greater than 0.5 meter but less than 1 meter. Similarly, the distance sensor 330 may generate the distance sensing signal Psense_1 to be high and Psense_0.5 and Psense_2 to be low if the sensed distance is greater than 1 meter but less than 2 meters, and Psense_2 to be high and Psense_0.5 and Psense_1 to be low if the sensed distance is greater than 2 meters.

When the sensed distance is greater than 0.5 meter but less than 1 meter (Psense_0.5 is high, and Psense_1 and Psense_2 are low), the switch N1 is turned on and the switches N2 and N3 are turned off, therefore current I1 flows while currents I2 and I3 are off. This effectively turns on one violet light LED and one infrared light LED as having a ratio of 1 to 1 (e.g., 1V 1IR). When the sensed distance is greater than 1 meter but less than 2 meters (Psense_1 is high, and Psense_0.5 and Psense_2 are low), the switch N2 is turned on and the switches N1 and N3 are turned off, therefore current I2 flows while currents I1 and I3 are off. This effectively turns on two violet light LEDs and two infrared lights LED while maintaining the ratio of 1 to 1 (e.g., 2V 2IR). When the sensed distance is greater than 2 meters (Psense_2 is high, and Psense_0.5 and Psense_1 are low), the switch N3 is turned on and the switches N1 and N2 are turned off, therefore current I3 flows while currents I1 and I2 are off. This effectively turns on four violet light LEDs and four infrared lights LED while maintaining the ratio of 1 to 1 (e.g., 4V 4IR).

According to some test results, the effectiveness of the cleaning, sanitization, and disinfection of the system 300 may diminish at a long distance, and it may be beneficial to increase the number of the violet and infrared lights for a long-distance application to increase the effectiveness of the cleaning, sanitization, and disinfection of the system 300.

In one embodiment, each of the power rating of the violet light LEDs and the infrared light LEDs may be substantially similar to each other, for example, 3 watts. In this case, the number of LEDs emitting light may vary depending on the distance. In another embodiment, the same number of LED lights may emit irrespective of the distance, but the power rating of the LED lights may vary depending on the distance. For example, one violet light LED and one infrared light LED may be turned on at a distance between 1 meter and 2 meters, and the power rating of the violet and infrared LED lights may be 12 watts. Also, one violet light LED and one infrared light LED may be turned on at a distance greater than 2 meters, and the power rating of the violet and infrared LED lights may be 24 watts.

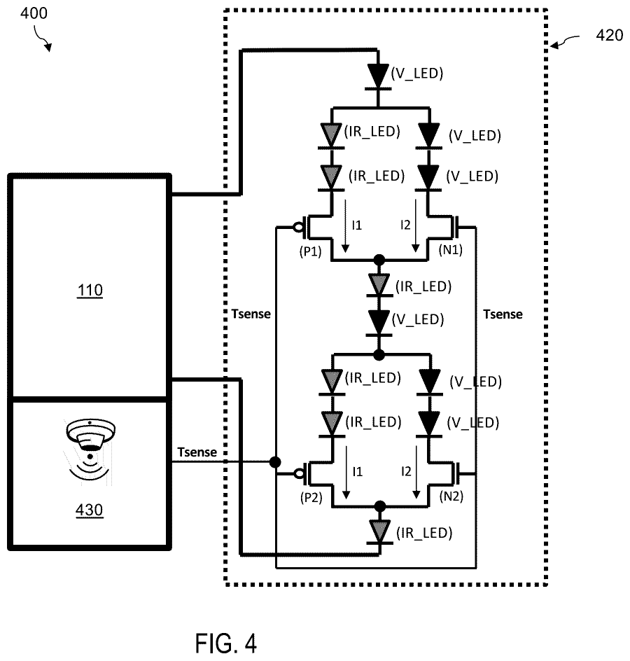

FIG. 4 shows an example of the present system, according to yet another embodiment. The system 300 includes a power supply 110, an array of light emitting diode (LED) lighting 320, and a temperature sensor 430. In the present example, the array of LED lighting 420 includes six violet light LEDs and six infrared light LEDs. The temperature sensor 430 may sense a temperature of an air surrounding a target object or a surface temperature of the target object and generate a temperature sensing signal Tsense. The temperature sensing signal Tsense may be high if the sensed temperature is greater than a certain threshold temperature (e.g., 25° C.) or low when the sensed temperature is less than the threshold temperature.

When the temperature sensing signal Tsense is low (e.g., the sensed temperature is less than 25° C.), switches P1 and P2 are turned on and the switches N1 and N2 are turned off, therefore current I1 flows while current I2 is off. This effectively connects one violet light LED, three infrared light LEDs, one more violet light LED, and three infrared light LEDs in series configuring the array of LED lighting 220 as having a ratio of 1 to 3 (e.g., 1V 3IR). When the temperature sensing signal Tsense is high (e.g., the sensed temperature is greater than 25° C.), switches P1 and P2 are turned off and the switches N1 and N2 are turned on, therefore current I2 flows while current I1 is off. This effectively connects three violet light LEDs, one infrared light LED, three more violet light LEDs, and one infrared light LED in series configuring the array of LED lighting 220 as having a ratio of 3 to 1 (e.g., 3V 1IR). Although the present example shows the configurability between 3 to 1 ratio and 1 to 3 ratio, it is understood that the system 300 is capable of dynamically switching between at least two different ratios of the violet light LEDs and the infrared light LEDs using the temperature sensor 430. In one embodiment, the entire infrared light LEDs may be turned off when the temperature is greater than a threshold temperature.

According to some test results, the side effect of temperature increase by the infrared light may tolerable at a lower temperature, therefore it may be beneficial to increase the relative ratio of the infrared light LEDs with respect to the violet light LEDs to be high at a lower temperature and decrease the ratio of the relative ratio of the infrared light LEDs with respect to the violet light LEDs to be low at a higher temperature thereby increasing the effectiveness of the cleaning, sanitization, and disinfection of the system 300 while reducing the side effect of temperature increase by the infrared light.

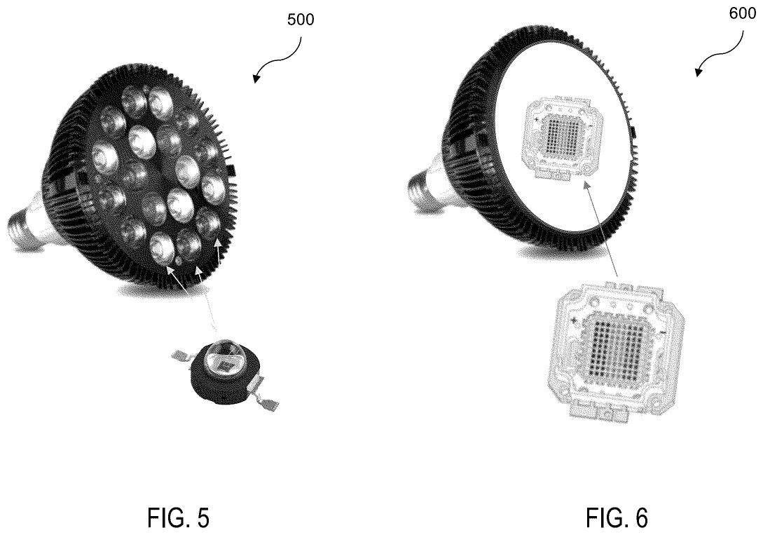

FIG. 5 shows an example of a light, according to one embodiment. The light 500 may include a plurality of individual chip-on-board (COB) diodes. Each of the COB diode may correspond to either a violet light LED or an infrared light LED. As a non-limiting example, the light 500 may include a total of 18 COB diodes of a specified ratio. Each of the COB diode may have a 3-watt power rating, therefore the light 500 may a power rating of 54 watt. The light 500 may correspond to any of the array of light emitting diode (LED) lighting 120, 220, 320, and 420 shown in FIGS. 1-4 without deviating from the scope of the present disclosure. The ratio of the violet light LEDs and the infrared light LEDs may be dynamically varied based on the user-configuration, operating conditions, and/or sensed signals as discussed with reference to FIGS. 1-4.

In one embodiment, the plurality of the COB diode is arranged in various forms and configurations, for example, a ceiling light, a wall light, a linear light, a dome light, a track light, a lamp, etc. The light 500 may have COB diodes arranged in circular fashion along at least one outer ring and one inner ring.

FIG. 6 shows an example of a light, according to another embodiment. The light 600 may include a single COB diode on which a plurality of individual violet and infrared LED elements of a specified ratio are formed. For example, the specified ratio of the violet and infrared LED elements may be 1 to 1, 2 to 1, 3 to 1, 4 to 1, 5 to 1, 3 to 2, 4 to 3, 5 to 3, etc. Depending on the specified ratio, the violet and infrared LED elements may be arranged in a matrix form having one or more rows and one or more columns. The number and order of the violet and infrared LED elements arranged in each row or column may vary without deviating from the scope of the present disclosure. Further, each of a group of the LED elements in the light 600 may be dynamically and selectively turned on or off using an internal switch (not shown). Similar to the light 500 shown in FIG. 5 the numbers and arrangements of the individual LED elements in the light 600 may vary based on the user-configuration, operating conditions, and/or sensed signals as discussed with reference to FIGS. 1-4.

In one embodiment, the array of light emitting diode (LED) lighting 120, 220, 320, and 420 shown in FIGS. 1-4 may include one or more single-COB lights 600 that are spaced from one another. The light 600 may provide uniform and higher density of light output while achieving more efficient use of surface space. The light 600 may provide wide and even spread of light over a large area. The configuration of the lights 500 and 600 may be modified to a small cubic shape to fit into an adequate small space, such as a shoes box, an appliance, a pet litter box, a refrigerator, or other appliances. The light 600 may have a micro-LED format that is feasible to be integrated into or coupled to a vacuum head, a cabinet space, a water filter system, a kitchen appliance, a refrigerator, or other space-limited applications.

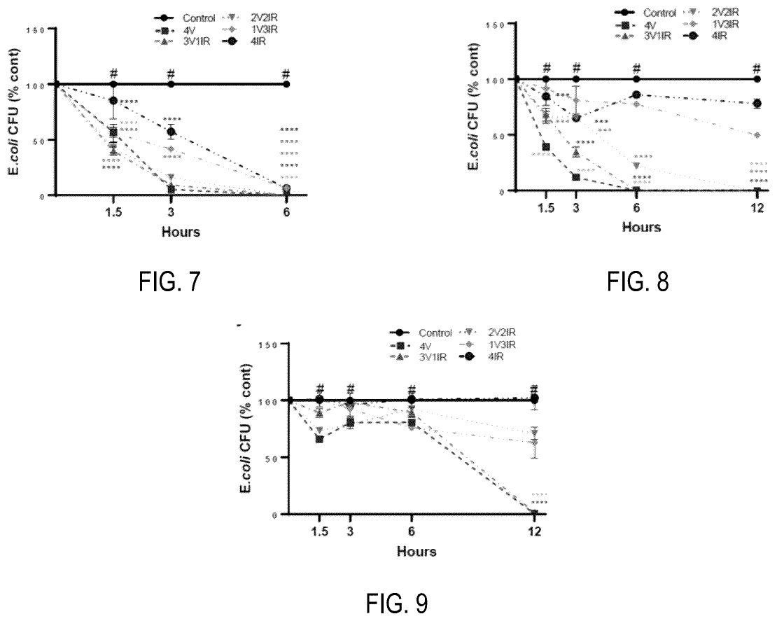

FIGS. 7-23 show test results of using various combinations of violet and infrared lights show relative effectiveness against bacterial and viral strains. For the purpose of comparative analyses, test results are shown for five different ratios of violet and infrared lights, namely, four violet lights (4V), three violet lights and one infrared light (3V-1IR), two violet lights and two infrared lights 2V-21R, one violet light and three infrared lights (1V-3IR), and four infrared lights (4IR).

The tests are performed with regular and MDR bacteria at various distances from the lighting, for example, three different distances at 0.5, 1, and 2 meters. Table 1 shows exposed light intensities calculated based on various distances from a combined light that is rated at 24 watts.

TABLE 1

| Distance | Exposed light intensity | Exposed |
|---|---|---|
| (meter) | (mW/cm2) | Area (m2) |
| 0.5 | 6.9 | 0.262 |
| 1 | 2.3 | 1.047 |
| 2 | 0.6 | 4.187 |

The combined light of the violet light LED that has a wavelength of 405 nm and the infrared light LED that has a wavelength of 850 nm is exposed at a power of 4.6 mW/cm² at 0.5 meter, 2.3 mW/cm² at 1 meter, and 0.6 mW/cm² at 2 meters, respectively. Both regular strains and MDR strains are tested for demonstrating effectiveness of cleaning, sanitizing, and disinfecting by the present system and method.

Some test results show effective suppression of bacterial growths using a combination of 405 nm (V) and 850 nm (IR) with a 3:1 ratio (i.e., 3V-1IR) as well as 4V compared to other light combinations. These test results demonstrate that more than 99% of bacteria is terminated under the exposure of combination of lights 3V-1IR or 4V within 6 hours at 0.5 or 1 meter in distance among the example of test MDR strains, such as E. coli (ATCC: BAA-2774), Salmonella enterica (ATCC: 19214) and Staphylococcus aureus (ATCC: BAA-1717), in addition to regular bacterial strains (E. coli & S. aureus). It is noted that these are merely examples of stains, and the present system and method may be applicable for sanitizing or killing other stains or bacteria without deviating from the scope of the present disclosure.

The 3V-1JR combined lights as well as 4V light are shown to effectively terminate Gram-negative and Gram-positive MDR strains at various distances, e.g., 50 cm, 1 meter and 2 meters.

Although some test results show an inverse correlation between effectiveness and distance, the combined light 3V-1IR and 4V effectively terminate bacteria even at 2 meters within 12-24 hours, depending on the bacterial strains. As an example, the combined light 3V-1IR or 4V light is shown to suppress nearly all bacteria (e.g., 99.99%) within 12 hours at 1 meter. The 3:1 ratio of the violet light LED and the infrared light LED may be broadly applied to clean, sanitize, or disinfect infectious diseases in a variety of spaces.

Since the differences of the effectiveness of bacterial growth suppression between 4V and 3V-1IR are subtle, either 3V-1IR or 4V may be used for sanitation, depending on an application. 3V-1IR may have a slightly higher potency, especially at a shorter distance (0.5 meter) due to dehydration by IR, although its effectiveness is nearly identical with 4V at 1 or 2 meters with some exceptions. Even though both 4V and the combined 3V-1JR light are substantially equally effective in terminating a variety of bacterial strains at various distances (50 cm, 1 and 2 meters), 3V-1IR may be used for a short distance application (e.g., less than 1 meter) and both 3V-1IR and 4V may be used for a longer distance application (e.g., greater than 1 meter). Mild heat generated by infrared (3V-1IR) may provide an extra dehydration effect for removing an odor. In case of applying the light in a cold room, refrigerator, or a cargo, or in temperature-sensitive application, the violet only light (e.g., 4V) without an infrared light may be adequately used due to a heat that may be generated by an infrared light. The 3V-1JR combined light may be used to sanitize or disinfect a variety of spaces including a lobby, an office, a bathroom, a hospital, a hotel, or many other locations with a heavy foot traffic, where infectious diseases often spread widely in a community.

For the regular bacterial strains and MDR-strains test shown in FIGS. 7-23, the density of the bacteria was initially measured at OD600, and a log-phased bacterial solution was diluted in LB broth to reach a constant density, i.e., OD=±1, and the bacteria is plated onto LB agar plates after calculation (e.g., 1/108 dilution). Each plate must receive the equal number of bacteria in triplicates (e.g., n=3/group in three independent experiments). The bacterial plates were then exposed to different lights and bacterial colonies are counted for 6 different time points (e.g., 0, 1.5, 3, 6, 12 or 24 hours) without a lid on. A control group was prepared simultaneously, but kept in the dark under the same conditions. At each time point, triplicate plates were removed after exposure for incubating colonies at 37° C. overnight. On the following day, bacterial colonies were counted in a blinded manner for analysis.

FIG. 7 shows test results of LED light exposure of 4V, 3V-1IR, 2V-21R, 1V-31R, and 4R to E. coli at a distance of 0.5 meter in 6 hours. Both 4V and 3V-1IR suppress more than 95% of viable E. coli survival at 0.5 meter within 3 hours. FIG. 8 shows test results of LED light exposure of 4V, 3V-1IR, 2V-21R, 1V-31R, and 4R to E. coli at a distance of 1 meter in 12 hours. Both 4V and 3V-1IR suppress more than 99% of viable E. coli survival at 1 meter within 6 hours. FIG. 9 shows test results of LED light exposure of 4V, 3V-1IR, 2V-21R, 1V-31R, and 4R to E. coli at a distance of 2 meters in 12 hours. Both 4V and 3V-1IR suppress more than 99% of viable E. coli survival at 2 meters within 12 hours.

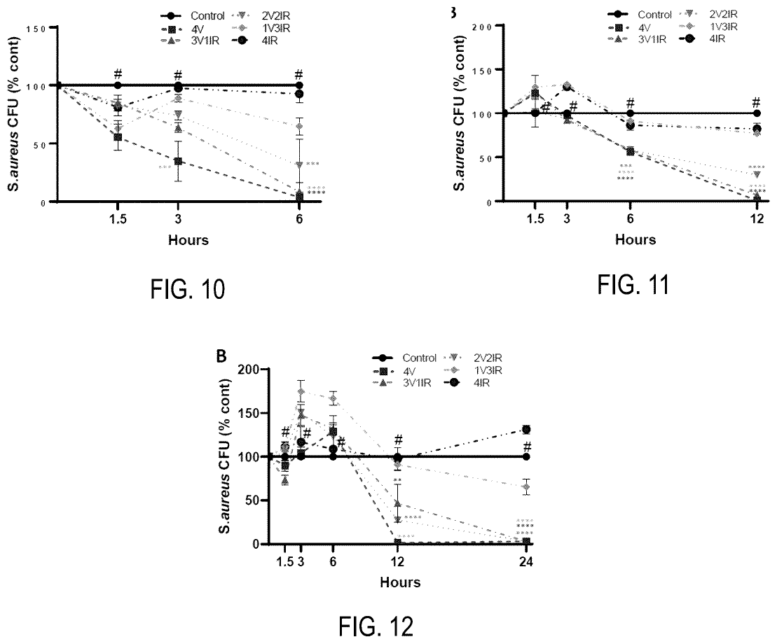

FIG. 10 shows test results of LED light exposure of 4V, 3V-1IR, 2V-21R, 1V-31R, and 4R to Staphylococcus aureus at a distance of 0.5 meter in 6 hours. Both 4V and 3V-1IR suppress more than 99% of viable Staphylococcus aureus survival at 0.5 meter within 6 hours. FIG. 11 shows test results of LED light exposure of 4V, 3V-1IR, 2V-21R, 1V-3IR, and 4R to Staphylococcus aureus at a distance of 1 meter in 12 hours. Both 4V and 3V-1IR suppress more than 99% of viable Staphylococcus aureus survival at 1 meter within 12 hours. FIG. 12 shows test results of LED light exposure of 4V, 3V-1IR, 2V-21R, 1V-31R, and 4R to Staphylococcus aureus at a distance of 2 meters in 24 hours. Both 4V and 3V-1IR suppress more than 99% of viable Staphylococcus aureus survival at 2 meters within 24 hours.

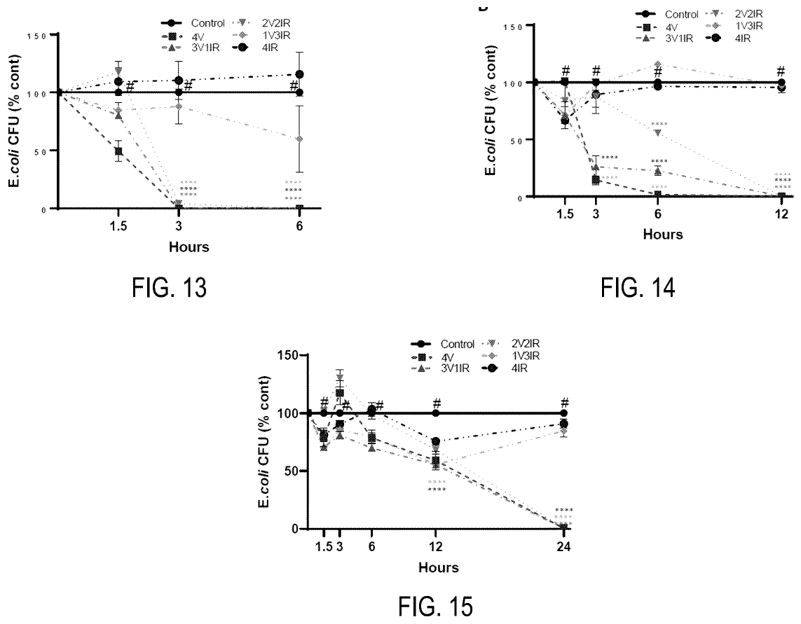

FIG. 13 shows test results of LED light exposure of 4V, 3V-1IR, 2V-21R, 1V-31R, and 4R to MDR-E. coli at a distance of 0.5 meter in 6 hours. Both 4V and 3V-1IR suppress more than 99% of viable MDR-E. coli survival at 0.5 meter within 3 hours. FIG. 14 shows test results of LED light exposure of 4V, 3V-1IR, 2V-21R, 1V-31R, and 4R to MDR-E. coli at a distance of 1 meter in 12 hours. Both 4V and 3V-1IR suppress more than 99% of viable MDR-E. coli survival at 1 meter within 6 hours. FIG. 15 shows test results of LED light exposure of 4V, 3V-1IR, 2V-2IR, 1V-3IR, and 4R to MDR-E. coli at a distance of 2 meters in 24 hours. Both 4V and 3V-1IR suppress more than 99% of viable MDR-E. coli survival at 2 meters within 24 hours.

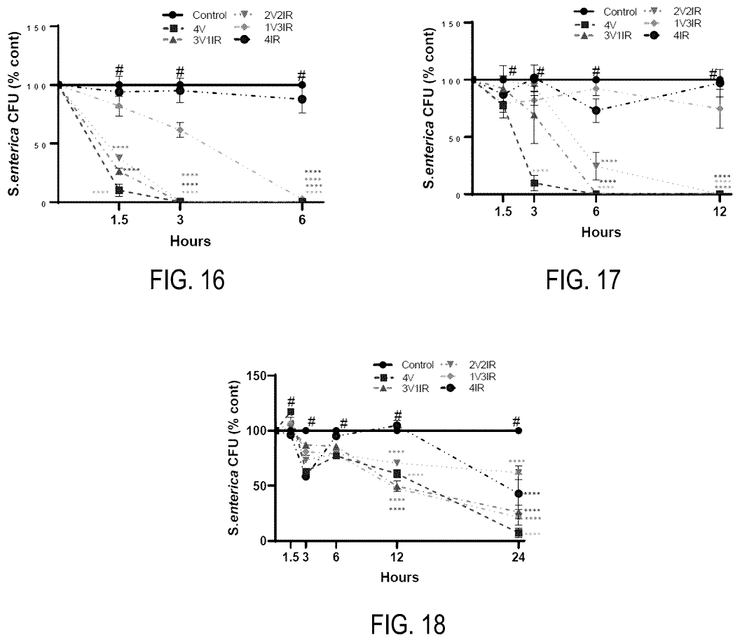

FIG. 16 shows test results of LED light exposure of 4V, 3V-1IR, 2V-2IR, 1V-3IR, and 4R to MDR-Salmonella enterica at a distance of 0.5 meter in 6 hours. Both 4V and 3V-1IR suppress more than 99% of viable MDR-Salmonella enterica survival at 0.5 meter within 3 hours. FIG. 17 shows test results of LED light exposure of 4V, 3V-1IR, 2V-2IR, 1V-3IR, and 4R to MDR-Salmonella enterica at a distance of 1 meter in 12 hours. Both 4V and 3V-1IR suppress more than 99% of viable MDR-Salmonella enterica survival at 1 meter within 6 hours. FIG. 18 shows test results of LED light exposure of 4V, 3V-1IR, 2V-21R, 1V-3IR, and 4R to MDR-Salmonella enterica at a distance of 2 meters in 24 hours. Both 4V and 3V-1IR suppress 74-93% of viable MDR-Salmonella enterica survival at 2 meters within 24 hours.

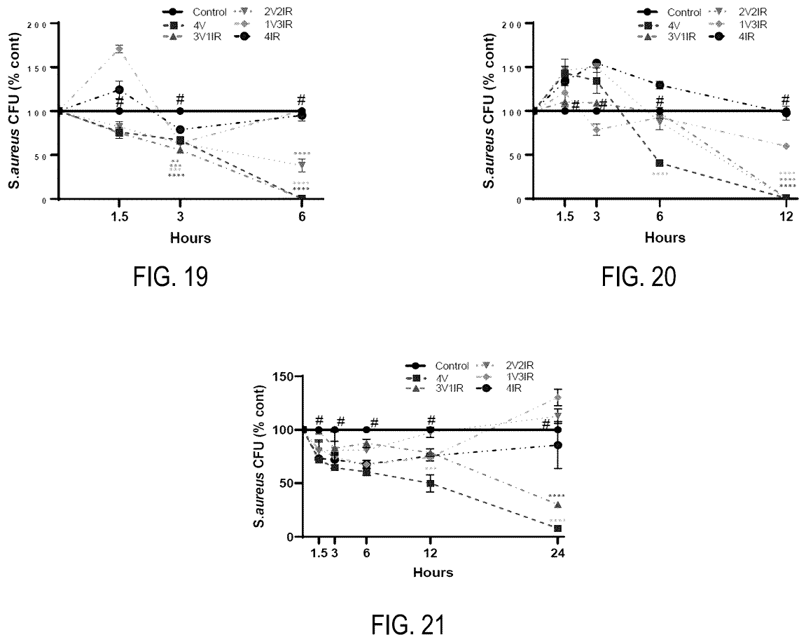

FIG. 19 shows test results of LED light exposure of 4V, 3V-1IR, 2V-21R, 1V-31R, and 4R to MDR-Staphylococcus aureus at a distance of 0.5 meter in 6 hours. Both 4V and 3V-1IR suppress more than 99% of viable MDR-Staphylococcus aureus survival at 0.5 meter within 6 hours. FIG. 20 shows test results of LED light exposure of 4V, 3V-1IR, 2V-21R, 1V-3IR, and 4R to MDR-Staphylococcus aureus at a distance of 1 meter in 12 hours. Both 4V and 3V-1IR suppress more than 99% of viable MDR-Staphylococcus aureus survival at 1 meter within 12 hours. FIG. 21 shows test results of LED light exposure of 4V, 3V-1IR, 2V-2IR, 1V-3IR, and 4R to MDR-Staphylococcus aureus at a distance of 2 meters in 24 hours. Both 4V and 3V-1IR suppress 70-92% of viable MDR-Staphylococcus aureus survival at 2 meters within 24 hours.

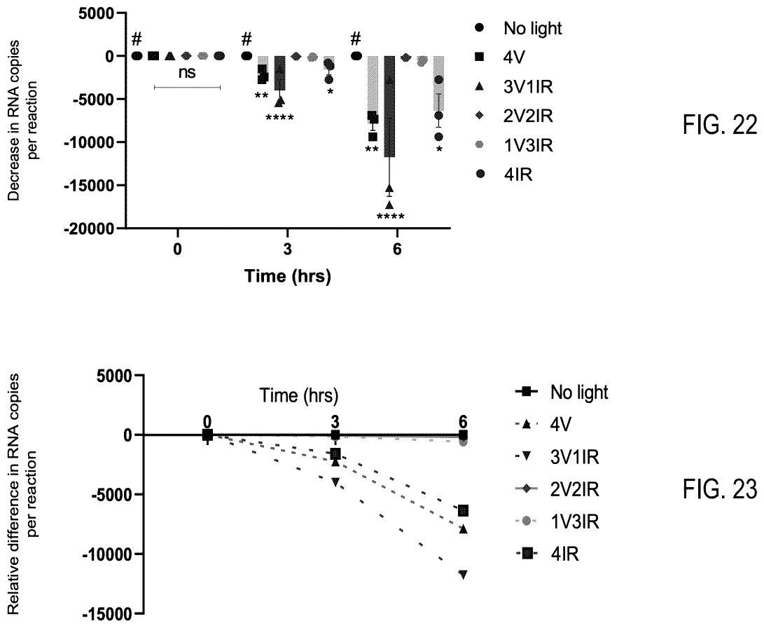

FIG. 22 shows test results of LED light exposure of 4V, 3V-1IR, 2V-2IR, 1V-3IR, and 4R to COVID-19 viral particles at a distance of 0.5 meter in 6 hours. FIG. 22 shows a decrease of RNA copies in reaction. FIG. 23 shows a relative difference in RNA copies in reaction for the same test results of FIG. 22.

During the COVID-19 tests, human saliva samples were exposed to the various combined lights (e.g., 4V, 3V-1IR, 2V-2IR, 1V-3IR and 4IR) at 0.5 meter for 3-6 hrs. After the isolation of intact viral particles using a COVID-19 isolation kit, the number of intact viral copies was assessed by quantitative real-time polymerase chain reaction (qRT-PCR) test. In the qRT-PCR test, the human COVID-19 saliva samples were diluted in phosphate buffered saline (PBS) solution at a 1:1 ratio and analyzed to count the number of intact COVID-19 viral particles.

The 3V-1IR is shown to be more effective other combinations of lights in terminating intact COVID-19 virus. The 3V-1IR exposure inflicts continual damage to COVID-19 virus as time progresses, resulting in a substantial reduction in the number of the viral copies. The reduction of the COVID-19 virus may be detected with a quantitative real-time test. Resultantly, the light exposure of the combined 3V-1IR for 3-6 hours may sufficiently suppress potential viral infection and effectively prevent virus-driven infectious diseases.

The present system and method may be used for cleaning, sanitizing, and disinfecting a surface of target objects for a variety of applications. The application of the present system and method may vary, depending on an amount of light exposure in a diverse indoor setting, for example, in a kitchen or a kitchen appliance as a wall or ceiling light, an internal light, or similar applications. The application of the present system and method may also include a wall/ceiling light or similar applications in a hallway of a building including school, hospital, and public buildings.

The present system and method may also be applied to clean, sanitize, and/or disinfect an appliance or a commonly-infected surface or space including, but not limited to, a refrigerator, a tank-top vehicle, a refrigerating truck, a freezer cargo van, a cargo truck, a cargo ship, a refrigerating container, a cold room, a trash can, a trash bin, a garbage disposal system, a dishwasher, a closet, a cabinet, a cupboard, a pantry, a drawer, an air conditioning/heating system, a ventilation duct, a vacuum cleaner, a robotic cleaning device, a pet litter box, a shoe box, a footwear sanitizing device, clothes, a water fountain, a water purifier, a drinking water filtration system, a sanitary application, a head gear, a helmet, a wrist protection gear and other personal protection gears, sports goods, ice-skates, socks, etc.

According to one embodiment, a system includes a power supply and a light coupled to the power supply and including a plurality of light-emitting diodes (LEDs). The plurality of LEDs includes a first LED that has a first wavelength and a second LED that has a second wavelength that is different from the first wavelength. A first number of the first LED is greater than a second number of the second LED.

The first wavelength may be a violet wavelength in a first wavelength range of 400-450 nm, and the second wavelength may be an infrared wavelength in a second wavelength range of 800-1000 nm.

A ratio of the first number and the second number may be three to one (3:1).

The system may further include a distance sensor that is configured to sense a distance from the light to a target object. The light may have a switch that is turned on or off based on the distance. A first subset of the plurality of LEDs may be turned on with the switch being turned on, and a second subset of the plurality of LEDs may be turned on with the switch being turned off. A first ratio of the first LED with respect to the second LED that are turned on with the switch being turned on may be different from a second ratio of the first LED with respect to the second LED that are turned on with the switch being turned off.

Light intensity of the light may be varied based on the distance sensed by the distance sensor.

The system may further include a temperature sensor that is configured to sense a temperature of a target object or a surrounding of the target object. The light may have a switch that is turned on or off based on the temperature. A first subset of the plurality of LEDs may be turned on with the switch being turned on, and a second subset of the plurality of LEDs may be turned on with the switch being turned off. A first ratio of the first LED with respect to the second LED that are turned on with the switch being turned on may be different from a second ratio of the first LED with respect to the second LED that are turned on with the switch being turned off.

The second LED may be turned off based on the temperature being greater than a threshold temperature.

Respective ones of the plurality of LEDs may have a substantially similar power rating.

Each of the plurality of LEDs may be a chip-on-board (COB) diode.

The light may include a single chip-on-board (COB) diode on which the plurality of LEDs is formed.

The light may be capable of emitting a combined light of the first wavelength and the second wavelength at a varying ratio based on an operating condition or an application.

According to another embodiment, a method for sanitizing a surface of a target object includes emitting a combined light of a first wavelength and a second wavelength. A first light intensity of a first LED that emits the first wavelength is greater than a second light intensity of a second LED that emits the second wavelength.

The first wavelength may be a violet wavelength in a first wavelength range of 400-450 nm, and the second wavelength may be an infrared wavelength in a second wavelength range of 800-1000 nm.

The combined light may be emitted by a plurality of LEDs including the first LED and the second LED, and wherein a first number of the first LED may be greater than a second number of the second LED.

A ratio of the first number and the second number may be three to one (3:1).

The method may further include: sensing a distance from a light source that emits the combined light toward the target object; turning on or off a switch of the light source based on the distance; and turning on a first number of the first LED and a second number of the second LED based on the distance using the switch.

The method may further include varying light intensity of the light source based on the distance.

The method may further include: sensing a temperature of a target object or a surrounding of the target object, turning on or off a switch of a light source that emits the combined light toward the target object based on the temperature; and turning on a first subset of the plurality of LEDs and a second subset of the plurality of LEDs based on the temperature using the switch. A first ratio of the first LED with respect to the second LED that are turned on with the switch being turned on may be different from a second ratio of the first LED with respect to the second LED that are turned on with the switch being turned off.

The second LED may be turned off based on the temperature being greater than a threshold temperature.

The first LED and the second LED may have a substantially similar power rating.

The above example embodiments have been described hereinabove to illustrate various embodiments of implementing a system and method for suppressing bacterial or viral growth using a combination of lights having at least two different wavelength spectra. Various modifications and departures from the disclosed example embodiments will occur to those having ordinary skill in the art. The subject matter that is intended to be within the scope of the invention is set forth in the following claims.

What is claimed is:

1. A system comprising:
a power supply; and
a light coupled to the power supply and comprising a plurality of light-emitting diodes (LEDs),
wherein the plurality of LEDs comprises a first LED that has a first wavelength and a second LED that has a second wavelength that is different from the first wavelength; and
wherein a first number of the first LED is greater than a second number of the second LED.

2. The system of claim 1, wherein the first wavelength is a violet wavelength in a first wavelength range of 400-450 nm, and the second wavelength is an infrared wavelength in a second wavelength range of 800-1000 nm.

3. The system of claim 2, wherein a ratio of the first number and the second number is three to one (3:1).

4. The system of claim 1, further comprising a distance sensor that is configured to sense a distance from the light to a target object,
wherein the light has a switch that is turned on or off based on the distance,
wherein a first subset of the plurality of LEDs is turned on with the switch being turned on, and a second subset of the plurality of LEDs is turned on with the switch being turned off, and
wherein a first ratio of the first LED with respect to the second LED that are turned on with the switch being turned on is different from a second ratio of the first LED with respect to the second LED that are turned on with the switch being turned off.

5. The system of claim 4, wherein light intensity of the light is varied based on the distance sensed by the distance sensor.

6. The system of claim 1, further comprising a temperature sensor that is configured to sense a temperature of a target object or a surrounding of the target object,
wherein the light has a switch that is turned on or off based on the temperature,
wherein a first subset of the plurality of LEDs is turned on with the switch being turned on, and a second subset of the plurality of LEDs is turned on with the switch being turned off, and
wherein a first ratio of the first LED with respect to the second LED that are turned on with the switch being turned on is different from a second ratio of the first LED with respect to the second LED that are turned on with the switch being turned off.

7. The system of claim 6, wherein the second LED is turned off based on the temperature being greater than a threshold temperature.

8. The system of claim 1, wherein respective ones of the plurality of LEDs have a substantially similar power rating.

9. The system of claim 1, wherein each of the plurality of LEDs is a chip-on-board (COB) diode.

10. The system of claim 1, wherein the light comprises a single chip-on-board (COB) diode on which the plurality of LEDs is formed.

11. The system of claim 1, wherein the light is capable of emitting a combined light of the first wavelength and the second wavelength at a varying ratio based on an operating condition or an application.

12. A method for sanitizing a surface of a target object comprising:
emitting a combined light of a first wavelength and a second wavelength,
wherein a first light intensity of a first LED that emits the first wavelength is greater than a second light intensity of a second LED that emits the second wavelength.

13. The method of claim 12, wherein the first wavelength is a violet wavelength in a first wavelength range of 400-450 nm, and the second wavelength is an infrared wavelength in a second wavelength range of 800-1000 nm.

14. The method of claim 13, wherein the combined light is emitted by a plurality of LEDs including the first LED and the second LED, and wherein a first number of the first LED is greater than a second number of the second LED.

15. The method of claim 14, wherein a ratio of the first number and the second number is three to one (3:1).

16. The method of claim 12, further comprising:
sensing a distance from a light source that emits the combined light toward the target object;
turning on or off a switch of the light source based on the distance; and
turning on a first number of the first LED and a second number of the second LED based on the distance using the switch.

17. The method of claim 16, further comprising varying light intensity of the light source based on the distance.

18. The method of claim 12, further comprising:
sensing a temperature of a target object or a surrounding of the target object,
turning on or off a switch of a light source that emits the combined light toward the target object based on the temperature; and
turning on a first subset of the plurality of LEDs and a second subset of the plurality of LEDs based on the temperature using the switch, and
wherein a first ratio of the first LED with respect to the second LED that are turned on with the switch being turned on is different from a second ratio of the first LED with respect to the second LED that are turned on with the switch being turned off.

19. The method of claim 18, wherein the second LED is turned off based on the temperature being greater than a threshold temperature.

20. The method of claim 12, wherein the first LED and the second LED have a substantially similar power rating.
